(19) United States
(12) Patent Application Publication
(10) Pub. No.: US 2020/0054245 A1
(43) Pub. Date: Feb. 20, 2020

(54) SYSTEM AND METHOD OF CANNABIS MONITORING

(71) Applicant: Dr. Andrew B. Cooper Medicine Professional Corporation, Caledon East (CA)

(72) Inventor: Andrew Brent Cooper, Caledon East (CA)

(21) Appl. No.: 16/542,427

(22) Filed: Aug. 16, 2019

Related U.S. Application Data
(60) Provisional application No. 62/719,578, filed on Aug. 17, 2018.

(51) Int. Cl. A61B 5/08; G06F 16/22; G16H 40/67; A61B 5/00

(52) U.S. Cl. CPC A61B 5/082; G06F 16/22; G16H 40/67; A61B 5/7235; A61B 5/0022; A61B 5/6898; A61B 5/746; A61B 5/002; A61B 5/4845

(57) ABSTRACT

A system for monitoring cannabis safety for a user comprising: an intake device communicatively coupled to a cannabis analysis subsystem via a network; wherein said intake device collects session data from the user and transmits said collected session data to the cannabis analysis subsystem; and said cannabis analysis subsystem performs analysis of said transmitted session data and communicates a reading to said user.

### FIELD OF THE INVENTION

The present disclosure relates to monitoring, for example, self-monitoring by users of cannabis.

### BRIEF SUMMARY

A system for monitoring cannabis safety for a user comprising: an intake device, such as a Bluetooth-enabled cannabis vaporizer, communicatively coupled to a cannabis analysis subsystem via a network; wherein said intake device collects session data from the user and transmits said collected session data, such as data pertaining to intensity of use, to the cannabis analysis subsystem; and said cannabis analysis subsystem performs analysis of said transmitted session data and communicates a reading to said user.

A method of monitoring cannabis safety for a user comprising collecting session data associated with a session from the user, such as data pertaining to active component potency and chemical composition of consumed cannabis products; analyzing said collected session data; and communicating a reading to the user based on said analyzing.

A system for cannabis monitoring by a user, wherein said system comprises an application installed on a mobile device associated with the user, wherein said mobile device is communicatively coupled to a network, and said application receives session data associated with a session from an intake device; enables the user to input data associated with the session; produces tracking data based on at least one of said inputted data, said received session data, and data received from one or more sensors on said mobile device; and transmits, via said network, said produced tracking data to at least one of a cannabis safety self-monitoring device, and a cannabis analysis subsystem.

A method of cannabis monitoring for a user comprising receiving, by a cannabis analysis subsystem, data associated with a user comprising breath analytic data, tracking data, and session data; storing, in a database, said data associated with the user; and processing, by said cannabis analysis subsystem, said received data to determine data corresponding to a usage history of said user, and an elimination pattern of said user.

The foregoing and additional aspects and embodiments of the present disclosure will be apparent to those of ordinary skill in the art in view of the detailed description of various embodiments and/or aspects, which is made with reference to the drawings, a brief description of which is provided next.

### BRIEF DESCRIPTION OF THE DRAWINGS

The foregoing and other advantages of the disclosure will become apparent upon reading the following detailed description and upon reference to the drawings.

FIG. 1 is an illustration of a system for monitoring cannabis safety for a user.

FIG. 2 is an illustration of an embodiment of a mobile device.

FIG. 2B illustrates the variation in concentration of THC and associated metabolites in the blood over time.

FIG. 3 is a detailed illustration of a cannabis analysis subsystem.

FIG. 4 is an illustration of an example process for the operation of a system for monitoring cannabis safety for a user.

While the present disclosure is susceptible to various modifications and alternative forms, specific embodiments or implementations have been shown by way of example in the drawings and will be described in detail herein. It should be understood, however, that the disclosure is not intended to be limited to the particular forms disclosed. Rather, the disclosure is to cover all modifications, equivalents, and alternatives falling within the spirit and scope of an invention as defined by the appended claims.

### DETAILED DESCRIPTION

Cannabis is used extensively for medical and recreational purposes. Many jurisdictions are legalizing or at least decriminalizing cannabis. For example, the Canadian Federal government has announced that cannabis will be legalized for recreational purposes in October 2018, and is already legalized for medical purposes. Many states in the United States have legalized the use of cannabis as well.

Currently, law enforcement agencies in many jurisdictions use qualitative and quantitative saliva based testing and blood testing to determine concentrations of active ingredients, such as
delta-9-tetrahydrocannabinol (THC),
cannabigerol (CBG),
cannabidiol (CBD),
cannabichromene (CBC),
cannabidivarin (CBDV),
cannabichromevarin (CBCV),
tetrahydrocannabivarin (THCV),
cannabigerivarin (CBGV),
cannabichromanon (CBCN),
cannabielsoin (CBE),
cannabifuran (CBF),
cannabicyclol (CBL),
cannabinol (CBN),
cannabinodiol (CBND),
cannabitriol (CBT),
cannabivarin (CBV),
other isocannabinoids,
other terpenes,
flavonoids, and
other related compounds.

Law enforcement agencies also use qualitative and quantitative saliva based testing and blood testing to determine concentrations of metabolites formed as a result of metabolism of cannabis by the body.

However this may be inadequate. For example, exhaled breath samples from users may be better than both saliva- and blood-based tests in determining oral THC concentrations, especially if users have ingested cannabis via edibles. Furthermore there may be a slower absorption rate of active ingredients for certain types of administration mechanisms. For example, active ingredients taken via edibles and beverages have slower absorption rates compared to vaping or smoking. Therefore, users may want to monitor the concentrations of active ingredients over a longer period of time, as it may take a longer time before concentrations peak for certain types of administration mechanisms. Some types of users, for example, patients taking cannabis for pain relief, may want to monitor the concentrations of certain active ingredients to ensure that they are taking sufficient levels of the desired active ingredients, but want to ensure that they avoid intake of psychoactive ingredients to avoid being impaired. Some users may also want to identify which combinations of cannabinoids result in unwanted impairment.

Different administration mechanisms may also result in the production of different metabolites. For example, oral administration may result in certain metabolites which are either not present or present in different quantities when compared to administration via the inhaled route.

There is a need for users to be able to self-monitor the intake of active ingredients and metabolites associated with cannabis to demonstrate the user's responsible compliance with applicable laws such as operating a conveyance of any kind or engaging in safety or performance-sensitive work activities. Furthermore, there is a need to do so from exhaled breath samples taken from a user. In monitoring, it is important to recognize that the effects of cannabis may vary significantly from individual to individual. Metabolites may themselves have an effect on users. Rates of conversion of active ingredients to metabolites vary amongst individuals. Furthermore, clearance rates vary amongst individuals based on, for example, body fat composition, recent exercise levels and genetic variance in enzyme activities across individuals. Also, there may be variation of active ingredients among crops for the same strain. There may also be synergistic effects when other psychoactive substances such as alcohol are consumed.

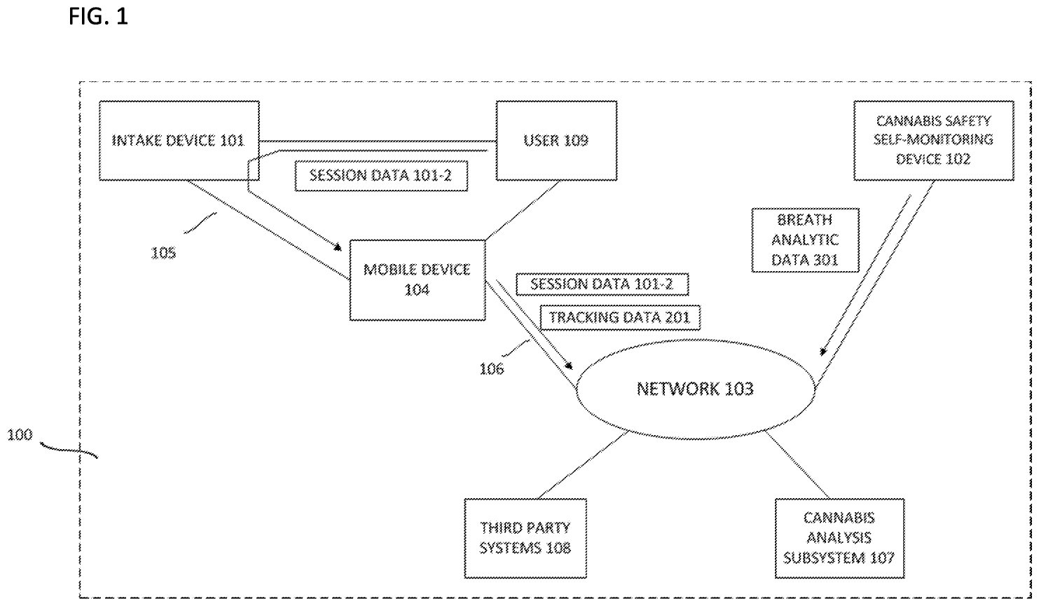

FIG. 1 shows an embodiment of a system 100 for monitoring cannabis safety for a user. In FIG. 1, intake device 101 is coupled to mobile device 104 via connection 105. Intake device 101 may be, for example, a Bluetooth-enabled cannabis vaporizer. Mobile device 104 and intake device 101 are associated with user 109. Mobile device 104 is coupled to cannabis analysis subsystem 107, third party systems 108 and cannabis safety self-monitoring device 102 via network 103 and connection 106.

System 100 represents one embodiment of a system for monitoring cannabis safety for a user. It would be known to those of skill in the art that other embodiments are also possible. For example, in some embodiments, intake device 101 is coupled to cannabis analysis subsystem 107 via a direct connection to network 103 or via a one-to-one communication technology such as Bluetooth. In other embodiments, intake device 101 is coupled to cannabis analysis subsystem 107 via cannabis safety self-monitoring device 102 and network 103. In yet other embodiments, intake device 101 is coupled to mobile device 104, which is then coupled to cannabis analysis subsystem 107 via a direct connection to network 103, via cannabis safety self-monitoring device 102 and network 103, or via a one-to-one communication technology such as Bluetooth. In yet other embodiments, intake device 101 includes embedded system circuitry on which cannabis analysis subsystem 107 resides. Also for example, in some embodiments, mobile device 104 is coupled to cannabis analysis subsystem 107 via a direct connection to network 103 or via a one-to-one communication technology such as Bluetooth. In yet other embodiments, mobile device 104 includes embedded system circuitry on which cannabis analysis subsystem 107 resides.

An example of the operation of system 100 will be explained below with reference to FIGS. 1-4.

Intake device 101 performs the function of breath topology analysis via for example hot wire anemometry and inhalation volume time and temperature time integration to create a measure of session intensity. For example, the Drager Spirolog® device may be used to perform hot wire anemometry, using a coaxial flow sensor, which may also include inspiratory airflow bypass and low temperature air/vapor past the sensor. Where the Spirolog device, for example, is used, there may be a data port that receives data such as a timestamp, flow as a function of time/session, and heating chamber (HC) temperature as a function of time/session. Also, where the Spirolog device, for example, is used, there may be at one end a high thermal conductivity vapor path connected to a mouthpiece, and there may be at another end a particulate filter high/detachment point (e.g., for connection to a mouthpiece). Cannabis safety self-monitoring device 102 performs the function of collecting inhaled and exhaled samples and analyzing these collected samples using, for example, a breathalyzer. The amounts of the one or more byproducts produced will depend on the concentration of the substance in the sample. Therefore by obtaining measures related to the properties or the amount of the one or more byproducts, the amount and concentration of the substance in the sample can be determined. An example of a breathalyzer device is the Cannabix Marijuana Breathalyzer produced by Cannabix Technologies Inc., which uses field asymmetric waveform ion mobility spectrometry (FAIMS) to detect THC in exhaled breath samples. The detection of cannabis and other controlled substances using FAIMS is described in, for example, Patent Cooperation Treaty (PCT) Application Publication No. WO/2017/147687 to Attariwala et al, with an international filing date of Mar. 6, 2017 and a publication date of Sep. 8, 2017.

In some embodiments, there are one or more breathalyzers to measure the amounts and concentrations of at least one of:

one or more active ingredients in the cannabis product, or

one or more metabolites formed as a result of metabolism of the cannabis.

In some embodiments, intake device 101 is combined with a device to enable administration of cannabis, for example, a vaporizer or “vape” device, a pipe or any other suitable implement to allow user 109 to inhale cannabis.

In some embodiments, in addition to the one or more active ingredients or one or more metabolites, the cannabis safety self-monitoring device 102 also analyzes for the presence of other psychoactive ingredients such as alcohol or other medications and illicit substances of interest. In these embodiments, there may be one or more breathalyzers and/or FAIMS configurations to measure for the presence of other psychoactive ingredients.

In some embodiments, intake device 101 is equipped to communicate with external networks, devices, systems, and subsystems using communication technologies known to those of skill in the art. This includes, for example, wired or wireless communications via protocols and technologies such as BLUETOOTH®, Wi-Fi, Near Field Communications (NFC), Radio Frequency Identification (RFID), 3G, Long Term Evolution (LTE), Universal Serial Bus (USB) and other protocols and technologies known to those of skill in the art.

Intake device 101 also comprises, for example, one or more flow sensors, temperature sensors and processors to detect and collect session data 101-2 for a user session. In some embodiments, intake device 101 allows users to input one or more portions of data as part of session data 101-2. Session data 101-2 may pertain generally to the intensity of use. Session data 101-2 can be, for example, data gathered from intake device 101, such as flow and temperature exports. Session data 101-2 can be, for example, product data input by a user into a mobile phone app or via a user interface on intake device 101. Session data 101-2 can be, for example, data collected by a FAIMS of intake device 102. Session data 101-2 may include one or more of:
Session start time,
Session end time,
Temperatures during the session,
Duration of heating of the cannabis product,
Flow rates during the session,
Data about the strain which is being inhaled,
Manufacturer and brand name of the cannabis product,
Nature of administration mechanism,
Amount consumed,
Type of product consumed,
Active component potency,
Chemical composition of consumed cannabis products,
Concentrations of active ingredients in inhaled samples,
Concentrations of active ingredients in exhaled samples,
Concentrations of metabolites in exhaled samples,
Concentrations of other psychoactive ingredients, for example, alcohol, and
Other information related to the above.

In some embodiments, the intake device 101 measures concentration of active ingredients as they are being inhaled, and includes this information in the session data 101-2.

In further embodiments, intake device 101 prompts the user to provide exhalation breath samples after one or more preset time periods have elapsed as part of session data 101-2. This enables time profiles of concentration of metabolites to be created and also enables measurement of:

conversion rates of active ingredients to metabolites, and

clearance of metabolites.

As shown in FIG. 1, the session data 101-2 detected and collected by intake device 101 is transmitted to coupled external devices and networks such as mobile device 104, network 103, cannabis safety self-monitoring device 102, cannabis analysis subsystem 107 and third party systems 108 via, for example connection 105.

Connections 105 and 106 are implemented using appropriate wired or wireless communications via protocols and technologies such as BLUETOOTH®, Wi-Fi, Near Field Communications (NFC), Radio Frequency Identification (RFID), 3G, Long Term Evolution (LTE), Universal Serial Bus (USB) and other protocols and technologies known to those of skill in the art.

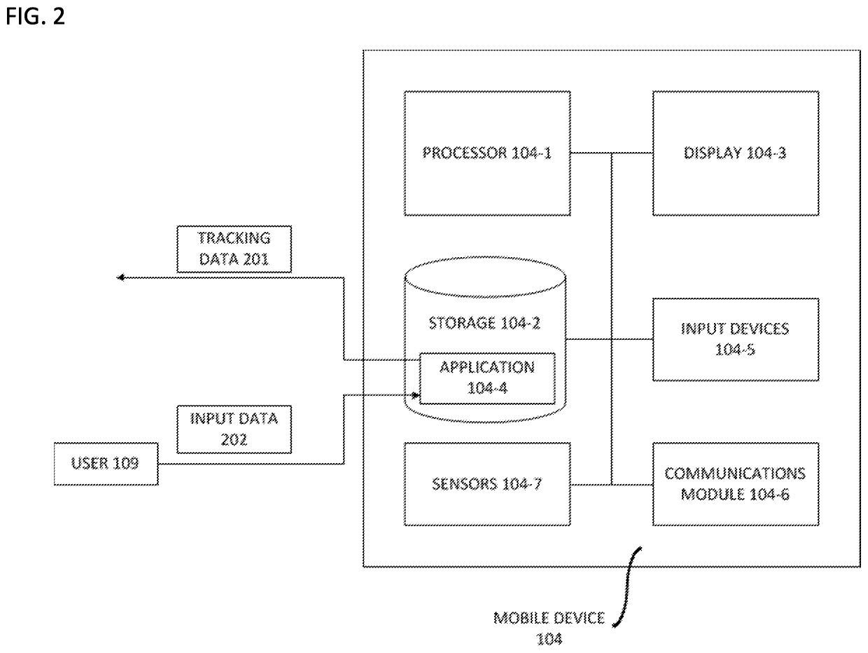

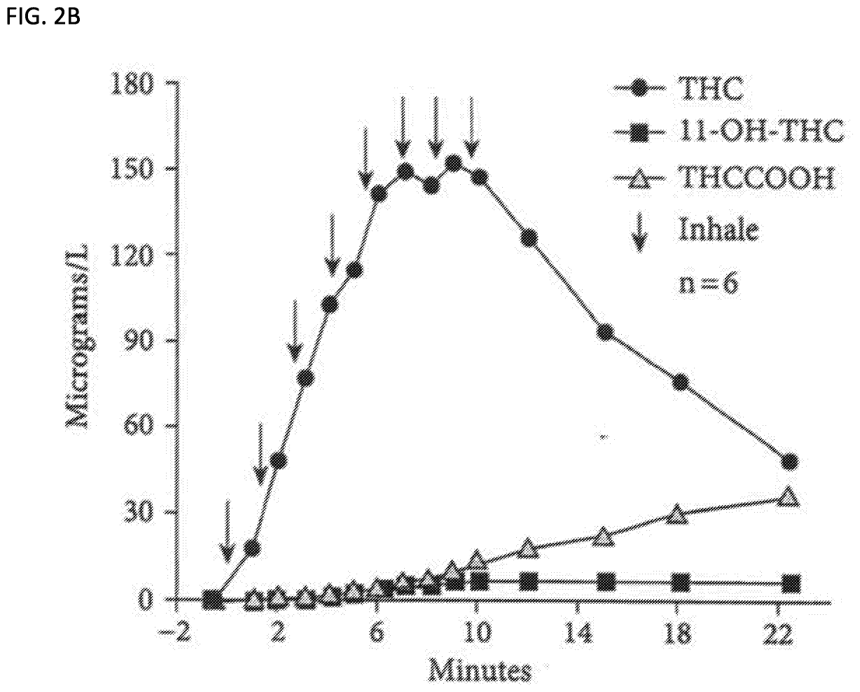

Mobile device 104 is, for example a smartwatch, smartphone, tablet, laptop, or any appropriate computing and network-enabled device. An embodiment of mobile device 104 is shown in FIG. 2. Processor 104-1 performs processing functions and operations necessary for the operation of mobile device 104, using data and programs stored in storage 104-2. An example of such a program is application 104-4 which will be discussed in more detail below. Display 104-3 performs the function of displaying data and information for user 109. Input devices 104-5 allow user 109 to enter information. This includes, for example, devices such as a touch screen, mouse, keypad, keyboard, microphone, camera, video camera and so on. In one embodiment, display 104-3 is a touchscreen which means it is also part of input devices 104-5. Communications module 104-6 allows mobile device 104 to communicate with devices and networks external to mobile device 104 via, for example connection 106. This includes, for example, wired or wireless communications via protocols and technologies such as BLUETOOTH®, Wi-Fi, Near Field Communications (NFC), Radio Frequency Identification (RFID), 3G, Long Term Evolution (LTE), Universal Serial Bus (USB) and other protocols and technologies known to those of skill in the art. Sensors 104-7 perform functions to sense or detect environmental or locational parameters. Sensors 104-7 include, for example, accelerometers, gyroscopes, magnetometers, barometers, Global Positioning System (GPS), proximity sensors and ambient light sensors. The components of mobile device 104 are coupled to each other as shown in FIG. 2.

Application or “app” 104-4 will now be discussed in more detail. In some embodiments, application or “app” 104-4 is a session tracking application. In some embodiments, app 104-4 presents a user interface to enable user 109 to input data 202 into application 104-4. Then, based on at least one of, for example:
input data 202,
session data 101-2, and
data received from sensors 104-7,
   app 104-4 produces tracking data 201 as shown in FIG. 2.

As shown in FIG. 1, in some embodiments, the tracking data 201 and detected session data 101-2 are transmitted by at least one of mobile device 104 and intake device 101 to at least one of cannabis safety self-monitoring device 102 and cannabis analysis subsystem 107 via, for example, network 103. In some of the embodiments where the tracking data is transmitted by mobile device 104, the transmission is performed by, for example, application 104-4. In further embodiments, by measuring the flow rates and the duration based on the session start and end times; and together with the inputted data 202, the application 104-4 determines an intensity of a session and includes data related to the determined intensity within tracking data 201.

In further embodiments, the application 104-4 enables the user to measure cognitive and behavior impairment during and after a session. For example, the application 104-4 may enable the user to self-assess measures of cognitive and motor function which are relevant to safe operation of a conveyance or performance of a safety sensitive activity, or otherwise approach the standards established for field determination of deemed impairment by law enforcement officers. These include, for example one or more psychophysical tests to challenge the user's coordination and ability to perform Divided Attention Tasks (DATs). Functionalities to measure cognitive and behavior impairment due to cannabis and other psychoactive ingredient ingestion have already been demonstrated in mobile device applications, as well as Cambridge Cognition's CANTAB cognitive research software. Relevant tests of impairment include, but are not limited to the following: (a) stop signal test; (b) tower of London test; (c) spatial memory tasks; (d) digit symbol substitution task; and (e) critical tracking task. For example, the DRUID® application (See, e.g. https://www.druidapp.com/about, retrieved Aug. 10, 2018) incorporates tasks that measure reaction time, decision making, Divided Attention Tasks (DATs), hand-eye coordination and balance. The AlcoGait and AlcoWear applications are able to detect the level of alcohol impairment using sensors in smartphones and smartwatches respectively. (See, e.g. http://web.cs.wpi.edu/˜emmanuel/research/projects/alcogait/, retrieved Aug. 10, 2018). In some embodiments, the outputs from sensors 104-7 are used by application 104-4 to perform these measurements of cognitive and behavior impairment. In some embodiments, data based on the results of these tests are included within tracking data 201.

There may be variants to the above. For example, in some embodiments, users input data into intake device 101, and this data is transmitted to either cannabis analysis subsystem 107 or cannabis safety self-monitoring device 102 via, for example, connection 106 and network 103.

Network 103 may be implemented in a variety of ways. For example, in some embodiments, network 103 comprises one or more subnetworks. In another embodiment, network 103 is implemented using one or more types of networks known to those of skill in the art. These types of networks include, for example, wireless networks, wired networks, Ethernet networks, local area networks, metropolitan area networks and optical networks. In some embodiments, network 103 comprises at least one of a private network or a public network.

Cannabis safety self-monitoring device 102 can be, for example, the “THC Breathalyzer” manufactured by Cannabix Technologies Inc., deployed in a hardened package for public place installation, such as a confidential self-assessment booth, and bundled with elements including but not limited to: a point of sale (POS) device, a user interface to a user account in cannabis analysis subsystem 107, and a hygienic single use breath collection dispenser. In some embodiments, cannabis safety self-monitoring device 102 comprises a breathalyzer for user 109 to breath into, and perform analysis of the user 109 breath samples together with other data to produce breath analytic data 301 as shown in FIG. 1. In further embodiments, cannabis safety self-monitoring device 102 is enabled to transmit and receive data using technologies known to those of skill in the art. This includes, for example, wired or wireless communications via protocols and technologies such as BLUETOOTH®, Wi-Fi, Near Field Communications (NFC), Radio Frequency Identification (RFID), 3G, Long Term Evolution (LTE), Universal Serial Bus (USB) and other protocols and technologies known to those of skill in the art. In some embodiments, the cannabis safety self-monitoring device 102 implements one or more sobriety algorithms to deliver to the user, at the point of interaction, a measure of the likelihood of legally significant impairment. Cannabis safety self-monitoring device 102 is configured to detect one or more concentrations of one or more active ingredients or metabolites using one or more exhaled breath samples provided by user 109. In some embodiments, cannabis safety self-monitoring device 102 comprises a compact mass spectrometer and other sensors known to those of skill in the art to perform said detection. Examples of such sensors include sensors which use field asymmetric waveform ion mobility spectrometry (FAIMS) to detect THC in exhaled breath samples while excluding or discriminating from unwanted ions in the human breath matrix. As previously explained, these sensors are described in, for example, Patent Cooperation Treaty (PCT) Application Publication No. WO/2017/147687 to Attariwala et al.

In some embodiments, cannabis safety self-monitoring device 102 performs regression analysis or uses other data analysis techniques. For example, the variation of concentration of THC and associated metabolites in the blood over time after change to smoking, inhalation through vaporization, or transmucosal absorption, follows a certain profile, which is characterized by one or more concentration parameters or oral and smoked active metabolites, including THC, 11-OH-THC, and THCCOOH. In some embodiments, these one or more concentration parameters are determined and obtained via regression analysis or other data analysis techniques. In some embodiments, machine learning is used to develop user-specific predictive models for clearance of metabolites and correlation with functional measures of impairment as detected by self-administered test routines such as DRUID or CANTAB.

The results of these analyses can be used to, for example, determine clearance profile parameters. Clearance profile parameters include, for example, elimination half-lives (e.g., t ½ beta) of one or more active ingredients or metabolites. These clearance profile parameters may differ based on administration mechanism. For example, the variation of concentration of active ingredients and metabolites over time differs based on product characteristics, route of administration, pharmacokinetic interactions, or pharmacogenetic heterogeneity in metabolism. For example, CBD and CBN may identify recent intake—in a previous study, CBD and CBN were not found after 1 hour in either matrix at a limit of quantification (LOQ) equal to 1.0 micrograms/L. In another study, plasma THC concentrations remained greater than 1 microgram/L for at least 1 day after daily cannabis smoking and also after cessation of multiple 20mg oral THC doses. In another study, it was estimated that 2-22 mg THC is necessary to produce pharmacological effects in humans. In another study, blood THC concentrations of frequent, long-term cannabis smokers persisted for multiple days after cannabis discontinuation. In some embodiments, if route of administration specific information concerning the administration mechanism is entered, then the clearance profile parameters can be determined.

In further embodiments, the tracking data 201 transmitted to cannabis safety self-monitoring device 102 comprises data obtained from tests of cognitive and behavior impairment. This test data is then correlated with, for example, the measured blood concentrations of the one or more active ingredients or metabolites and the route-specific clearance profile parameters to determine the impact on the user. In further embodiments, these correlations are updated over time based on changes in user characteristics.

Breath analytic data 301 transmitted by cannabis safety self-monitoring device 102 includes, for example:
Data based on the user input concentration parameters,
Data based on the determined clearance profile parameters,
Data obtained from the determined impact on the user (e.g., from DRUID or CANTAB) based on the correlation of the blood concentrations of the one or more active ingredients or metabolites, and
Data based on analysis of the user 109 breath samples together with other data.

In some embodiments, the breath analytic data 301 is transmitted to the cannabis analysis subsystem 107, along with the tracking data 201 and detected session data 101-2. In further embodiments, cannabis safety self-monitoring device 102 detects the presence of other impairing substances such as alcohol, pharmaceutical medications, or illicit substances of interest. It is known to those of skill in the art that there are synergistic effects due to the combination of alcohol and cannabis. One such example is the presence of any amount of alcohol in conjunction with THC that may lead to significant impairment in user 109 capabilities.

Cannabis safety self-monitoring device 102 may be located in different locations. In some embodiments, cannabis safety self-monitoring device 102 is located in the home of user 109. In some embodiments, cannabis safety self-monitoring device 102 is located in a public facility, such as a car park, gymnasium, supermarket, medical facility, jurisdictionally allowed place of public cannabis consumption, and so on.

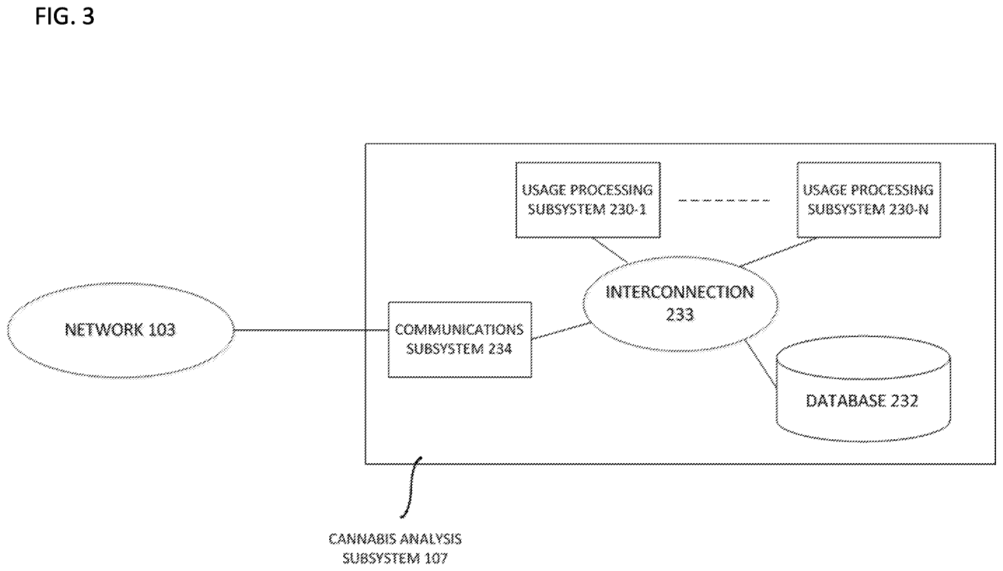

A detailed illustration of cannabis analysis subsystem 107 is shown in FIG. 3. Cannabis analysis subsystem 107 performs analysis of, for example, the breath analytic data 301, tracking data 201 and detected session data 101-2 received from the network to determine data corresponding to user 109 usage history and elimination patterns. These include parameters such as personalized active ingredient and metabolite clearance data. Cannabis analysis subsystem 107 is described in more detail in FIG. 3. In FIG. 3, communications subsystem 234 is coupled to network 103. Communications subsystem 234 receives information from, and transmits information to network 103.

Database 232 stores information and data for use by cannabis analysis subsystem 107. This includes, for example,
received breath analytic data 301,
tracking data 201,
detected session data 101-2,
data corresponding to user 109 usage history and elimination patterns,
one or more algorithms and programs necessary to perform processing of received data, and
other user data as needed.

In one embodiment, database 232 further comprises a database server. The database server receives one or more commands from, for example, usage processing subsystem 230-1 to 230-N and communication subsystem 234, and translates these commands into appropriate database language commands to retrieve and store data into database 232. In one embodiment, database 232 is implemented using one or more database languages known to those of skill in the art, including, for example, Structured Query Language (SQL). In a further embodiment, database 232 stores data for a plurality of users. Then, there may be a need to keep the set of data related to each user separate from the data relating to the other users. In some embodiments, database 232 is partitioned so that data related to each user is separate from the other users. In some embodiments, each user has an account with a login and a password or other appropriate security measures to ensure that they are only able to access their data, and unauthorized access of their data is prohibited. In a further embodiment, when data is entered into database 232, associated metadata is added so as to make it more easily searchable. In a further embodiment, the associated metadata comprises one or more tags. In yet another embodiment, database 232 presents an interface to enable the entering of search queries. In some embodiments, the data stored within database 232 is encrypted for security reasons. In further embodiments, other privacy-enhancing data security techniques are employed to protect database 232.

Usage processing subsystems 230-1 to 230-N perform processing and analysis within cannabis analysis subsystem 107 using one or more algorithms and programs residing on cannabis analysis subsystem 107. These algorithms and programs are stored in, for example,
database 232 as explained above, or
within usage processing subsystems 230-1 to 230-N.
   Examples of processing performed by usage processing subsystem 230-1 to 230-N include:
Implementations of algorithms used in processing of the received breath analytic data, tracking data 201 and detected session data 101-2 to determine data corresponding to user 109 usage history and elimination patterns, including parameters such as personalized drug clearance data;
Determining whether the user 109 can legally drive (or has imputed blood THC levels in relation to offence categories in Bill C-46);
Determining blood equilibration; and
Optimizing pain or symptom management for user 109.

As explained previously, active ingredients taken via different administration mechanisms have different conversion rates. In some embodiments, algorithms to calculate clearance and conversion rates take administration mechanisms into account.

Interconnection 233 connects the various components of cannabis analysis subsystem 107 to each other. In one embodiment, interconnection 233 is implemented using, for example, network technologies known to those in the art. These include, for example, wireless networks, wired networks, Ethernet networks, local area networks, metropolitan area networks and optical networks. In one embodiment, interconnection 233 comprises one or more subnetworks. In another embodiment, interconnection 233 comprises other technologies to connect multiple components to each other including, for example, buses, coaxial cables, USB connections and so on.

Various implementations are possible for cannabis analysis subsystem 107 and its components. In one embodiment, cannabis analysis subsystem 107 is implemented using a cloud-based approach. In another embodiment, cannabis analysis subsystem 107 is implemented across one or more facilities, where each of the components are located in different facilities and interconnection 233 is then a network-based connection. In a further embodiment, cannabis analysis subsystem 107 is implemented within a single server or computer. In yet another embodiment, cannabis analysis subsystem 107 is implemented in software. In another embodiment, cannabis analysis subsystem 107 is implemented using a combination of software and hardware.

As explained previously, elimination and clearance rates of active ingredients and metabolites will differ from individual to individual. It is therefore important, to measure personalized clearance rates for individuals.

In further embodiments, the cannabis analysis subsystem 107 receives either data obtained from tests of cognitive and behavior impairment, or data based on analysis of the results of tests of cognitive and behavior impairment. This data may be received as part of, for example, tracking data 201, or breath analytic data 301. In some embodiments, cannabis analysis subsystem 107 performs further analyses using this data. These analyses may include the previously detailed correlation of this data with, for example, the measured blood concentrations of the one or more active ingredients or metabolites and the clearance profile parameters to determine the impact on the user. In further embodiments, as previously explained, such correlations are updated over time based on changes in user characteristics such as physiological characteristics.

There may be many strains (e.g., chemovars and cultivars) of cannabis available, which may have independent and/or unique therapeutic indices balancing beneficial and undesired effects. Therefore there is a need for intensive processing and storage capabilities so as to adequately perform monitoring. By moving the processing functionality to cannabis analysis subsystem 107, this increases the processing power, storage capacity and information aggregation capability of system 100, and allows for more detailed analyses to be performed. In some embodiments, this enables the use of “big data” or detailed statistical processing techniques to enable monitoring.

In some embodiments, based on said processing and analysis performed above, cannabis analysis subsystem 107 transmits one or more readings (or personalized medicine reports) to user 109 via, for example application 104-4 residing on mobile device 104. In some embodiments, cannabis analysis subsystem 107 transmits one or more safety advisory alerts to user 109 via, for example application 104-4 residing on mobile device 104.

In some embodiments, application 104-4 retrieves data related to user 109 usage history and elimination data, such as personalized drug clearance data, from cannabis analysis subsystem 107. Then this data together with the session data received from intake device 101 and the tracking data, application 104-4 performs elimination analyses and produces readings and alerts for user 109.

In yet other embodiments, the breath analytic data, tracking data and session data is not transmitted to analysis subsystem 107 by cannabis safety self-monitoring device 102, allowing the user 109 to opt out if not comfortable with this data being stored in a cloud or centralized server for privacy and confidentiality reasons, and if wanting to retain control over this data. Then, in some of these embodiments, cannabis safety self-monitoring device 102 incorporates some or all of the processing capabilities of usage processing subsystems 230-1 to 230-N to perform the required analyses.

Third party systems 108 comprise systems which perform other functions. These may include, for example, ridesharing or taxi dispatch services, personal fitness databases, medical databases, insurance databases and so on. In some embodiments, the components of the system 100 are able to communicate with third party systems 108 to perform various functions. For example, if the user 109 is deemed impaired due to the tests and analyses performed by, for example, application 104-4, cannabis analysis subsystem 107 or cannabis safety self-monitoring device 102; then application 104-4, cannabis analysis subsystem 107 or cannabis safety self-monitoring device 102 is able to contact a ridesharing service or a taxi dispatch service which is part of third party system 108, so as to facilitate the user 109′s responsible decision not to operate a conveyance or to engage in safety or performance sensitive activity if self-monitoring data indicates a high likelihood of significant impairment of executive function. In some embodiments, third party systems 108 are provided with data concerning users by, for example, cannabis analysis subsystem 107.

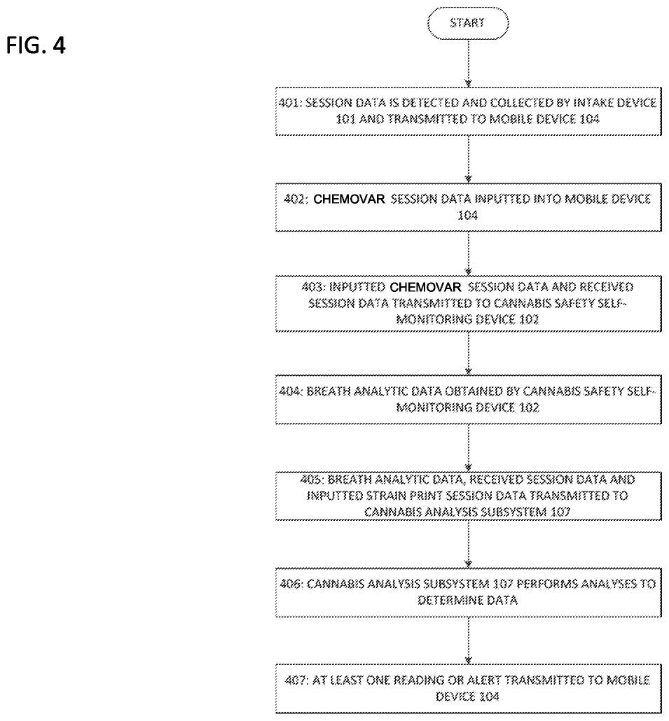

FIG. 4 demonstrates an example process of the operation of system 100. In step 401, session data connected to a user session such as session data 101-2 is detected and collected by intake device 101 and transmitted to, for example, application 104-4 of mobile device 104 as detailed above.

In step 402, user 109 inputs data such as data 202 into application 104-4 of mobile device 104, as detailed above. Then in some embodiments, within this step application 104-4 determines the intensity of the user session. Application 104-4 then produces tracking data such as tracking data 201 as described above.

In step 403, the received session data and tracking data is transmitted to cannabis safety self-monitoring device 102, as detailed above.

In step 404, breath analytic data is obtained by cannabis safety self-monitoring device 102 based on one or more exhaled samples provided by user 109, using the processes detailed above.

In step 405, the received session data, tracking data and obtained breath analytic data is transmitted to cannabis analysis subsystem 107. This data is stored in, for example, database 232 of cannabis analysis subsystem 107, as explained above

In step 406, further analyses as explained above are performed. In some embodiments these further analyses are performed to determine data corresponding to user 109 usage history and elimination patterns. In some embodiments, these further analyses are based on already stored data corresponding to user 109 usage history and elimination patterns.

In step 407, based on these further analyses, at least one reading or alert is transmitted to mobile device 104 as explained above.

Variations to the above process demonstrated in FIG. 4 are possible. For example, in some embodiments, after steps 401 and 402 have been performed, application 104-4 may retrieve usage history and elimination data from cannabis analysis subsystem 107 and perform further analyses to provide alerts or readings for user 109. In other embodiments, after step 401 and 402 have been performed, the received session data and tracking data is transmitted to cannabis analysis subsystem 107 where further analyses are performed.

Although the algorithms described above including those with reference to the foregoing flow charts have been described separately, it should be understood that any two or more of the algorithms disclosed herein can be combined in any combination. Any of the methods, algorithms, implementations, or procedures described herein can include machine-readable instructions for execution by: (a) a processor, (b) a controller, and/or (c) any other suitable processing device. Any algorithm, software, or method disclosed herein can be embodied in software stored on a non-transitory tangible medium such as, for example, a flash memory, a CD-ROM, a floppy disk, a hard drive, a digital versatile disk (DVD), or other memory devices, but persons of ordinary skill in the art will readily appreciate that the entire algorithm and/or parts thereof could alternatively be executed by a device other than a controller and/or embodied in firmware or dedicated hardware in a well-known manner (e.g., it may be implemented by an application specific integrated circuit (ASIC), a programmable logic device (PLD), a field programmable logic device (FPLD), discrete logic, etc.). Also, some or all of the machine-readable instructions represented in any flowchart depicted herein can be implemented manually as opposed to automatically by a controller, processor, or similar computing device or machine. Further, although specific algorithms are described with reference to flowcharts depicted herein, persons of ordinary skill in the art will readily appreciate that many other methods of implementing the example machine readable instructions may alternatively be used. For example, the order of execution of the blocks may be changed, and/or some of the blocks described may be changed, eliminated, or combined.

It should be noted that the algorithms illustrated and discussed herein as having various modules which perform particular functions and interact with one another. It should be understood that these modules are merely segregated based on their function for the sake of description and represent computer hardware and/or executable software code which is stored on a computer-readable medium for execution on appropriate computing hardware. The various functions of the different modules and units can be combined or segregated as hardware and/or software stored on a non-transitory computer-readable medium as above as modules in any manner, and can be used separately or in combination.

While particular implementations and applications of the present disclosure have been illustrated and described, it is to be understood that the present disclosure is not limited to the precise construction and compositions disclosed herein and that various modifications, changes, and variations can be apparent from the foregoing descriptions without departing from the spirit and scope of an invention as defined in the appended claims.

1. A system for monitoring cannabis safety for a user comprising:
an intake device communicatively coupled to a cannabis analysis subsystem;
wherein said intake device collects session data from the user and transmits said collected session data to the cannabis analysis subsystem; and
said cannabis analysis subsystem performs analysis of said transmitted session data and communicates a personalized report to said user based on the analysis.

2. The system of claim 1, wherein said cannabis safety self-monitoring device is configured to detect one or more concentrations of one or more metabolites from one or more exhaled samples provided by the user.

3. The system of claim 1, wherein said cannabis analysis subsystem comprises at least one usage processing subsystem coupled to a database;
wherein said database stores data corresponding to said user's usage history and elimination patterns; and
said analysis is performed based on said user's usage history and elimination patterns.

4. The system of claim 2, wherein said cannabis safety self-monitoring device performs regression analysis to determine at least one metabolite elimination t ½beta.

5. The system of claim 2, wherein said cannabis safety self-monitoring device implements one or more sobriety algorithms.

6. The system of claim 1, wherein
said intake device is communicatively coupled to a mobile device associated with said user;
said mobile device is communicatively coupled to said mobile device; and
said cannabis analysis subsystem communicates said personalized report to said mobile device.

7. The system of claim 6, wherein an application is installed on said mobile device;
said intake device transmits said collected session data to said mobile device;
said user inputs data to said application;
one or more sensors from said mobile device receives sensor data; and
said application produces tracking data based on at least one of said inputted data, said collected session data, and said sensor data.

8. The system of claim 7, wherein said application determines intensity of a session based on
said received session data, and
said inputted data.

9. The system of claim 7, wherein said mobile device is coupled to said cannabis analysis subsystem via a cannabis safety self-monitoring device; and
said cannabis safety self-monitoring device is configured to detect one or more concentrations of one or more metabolites from one or more exhaled samples provided by said user.

10. The system of claim 8, wherein
said application retrieves data corresponding to said user's usage history and elimination patterns from said cannabis analysis subsystem; and
said application performs elimination analysis and produces readings and alerts for the user.

11. A method of monitoring cannabis safety for a user comprising
collecting session data associated with a session from the user;
analyzing said collected session data; and
communicating a reading to the user based on said analyzing.

12. The method of claim 11, further comprising
producing tracking data based on at least one of
data inputted by a user,
said collected session data, and
data received by one or more sensors from a mobile device associated with said user; and
said analyzing comprises performing processing using said tracking data.

13. The method of claim 12, further comprising
producing breath analytic data based on one or more exhaled samples provided by the user; and
said analyzing comprises performing processing using said breath analytic data.

14. The method of claim 11, further comprising
storing data corresponding to the user's usage history and elimination patterns; and
said analyzing is performed based on said stored data.

15. The method of claim 12, further comprising determining an intensity of the session based on
said collected session data, and
said inputted data.

16. The method of claim 15, further comprising
retrieving data corresponding to said user's usage history and elimination patterns via said network;
said analyzing comprising performing elimination analysis; and
communicating an alert to said user based on said analyzing.

17. The method of claim 13, further wherein
prior to said analyzing, transmitting said collected session data, tracking data and breath analytic data to a cannabis analysis subsystem; and
said cannabis analysis subsystem performs said analyzing.

18. The method of claim 17, further wherein said analyzing comprises determining data corresponding to the user's usage history and elimination patterns.

19. The method of claim 18, wherein said cannabis analysis subsystem performs said communicating.

20. The method of claim 17, further wherein
said collecting of session data is performed by an intake device;
said method further comprises transmitting said collected session data to an application running on a mobile device associated with said user;
said producing of tracking data is performed by said application;
said method further comprises transmitting said collected session data and said tracking data to a cannabis safety self-monitoring device;
said producing of breath analytic data is performed by said cannabis safety self-monitoring device; and
said cannabis safety self-monitoring device performs said transmitting of said collected session data, tracking data and breath analytic data to said cannabis analysis subsystem.

21. The method of claim 20, wherein said tracking data comprises one or more results from measuring cognitive and behavior impairment.

22.-34. (canceled)
